(19) United States
(12) Patent Application Publication
(10) Pub. No.: US 2022/0220162 A1
(43) Pub. Date: Jul. 14, 2022

(54) TARGETED DELIVERY OF THE ABRIN-A A-CHAIN TO CANCER CELLS

(71) Applicants: Houshang Alizadeh, Karaj (IR); Fariba Rafiei, Kohgiloyeh (IR); Ali-Akbar Shahnejat Bushehri, Karaj (IR)

(72) Inventors: Houshang Alizadeh, Karaj (IR); Fariba Rafiei, Kohgiloyeh (IR); Ali-Akbar Shahnejat Bushehri, Karaj (IR)

(21) Appl. No.: 17/148,227

(22) Filed: Jan. 13, 2021

(51) Int. Cl. C07K 14/415; C12N 9/24; C12N 9/64; C12N 9/96; A61P 35/00; C07K 16/16

(52) U.S. Cl. CPC C07K 14/415; C12N 9/2497; C12N 9/6454; C12Y 304/21075; A61K 38/00; A61P 35/00; C07K 16/16; C07K 2319/33; C12N 9/96

(57) ABSTRACT

A targeted delivery of recombinant therapeutic protein to inhibit the growth of cancer cells is disclosed. The recombinant protein derived from Abrin-a A-chain able to inhibit the growth of cancer cell and conjugated to stxB to form Abrin-f-stxB. The abrin toxin A chain and the Shiga toxin B chain are linked with the furin linker to form a recombinant therapeutic protein as chemotherapeutic agent to inhibit the growth of cancer cells. A conjugate for the delivery of a compound into cancer cells, comprising a first module mediates cell targeting and facilitates cellular uptake, a second module facilitates transport to the endoplasmic reticulum (ER), a third module mediates translocation from the ER to the cytosol and a compound that is desired to be delivered to the cytosol. The recombinant protein Abrin-f-stxB exhibits high binding affinity of stxB as a drug delivery system to Gb3 receptor in targeted cancer diagnosis and treatment.

### BACKGROUND OF THE INVENTION

Cancer is one of the most significant diseases confronting mankind, and much research effort is going into its treatment. Many types of cancer are difficult to prevent. Despite several years of medical research, cancer is the second leading cause of death worldwide after cardiovascular disease. The global cancer burden is estimated to have risen to 27 million new cases and 16 million deaths worldwide by 2040.

There are different conventional treatment methods available to treat and manage cancer. Surgery, chemotherapy, and radiotherapy are some of the traditional and most widely used treatment options. Chemotherapy is one of the most effective treatments for many types of cancers. This method involves the use of powerful chemicals used as drug treatments to kill fast-growing cells. However, its efficacy and success are challenged due to the side effects. Some of the more modern strategies include hormone-based therapy, anti-angiogenic methods, stem cell therapies, and dendritic cell-based immunotherapy. Although many anti-cancer therapies are available, finding a cure for cancer continues to be a difficult task.

Therefore, many efforts have been made to develop more effective treatments, such as chemotherapy based on a new class of tumor-specific products that are produced using recombinant DNA technology. The recombinant proteins in anticancer are used with the main objectives of killing the tumor and cancer cells. Since anticancer drugs are often very toxic and have serious side effects, a common way to reduce side effects is to use the drug delivery systems to target the tumor site without leaking out into other sites and thereby reducing the exposure of powerful drugs to healthy tissues. Drug delivery in cancer therapy is crucial for optimizing the effect of drugs and reducing toxic side effects.

In the recent past, rapid progress in understanding the molecular biology of cancer cells has made a major impact on the design and engineering of immunotoxins as therapeutic proteins. Among these, there are recombinant or fusion toxins containing a toxin fused to an antibody or small molecules constructed by genetic engineering techniques. In several cancer cells, there is up-regulation of tumor-associated antigens and specific cell-surface receptors, which can be targeted with immunotoxins that this not only ensures site-specific delivery of the therapeutic molecules but also maximizes the effect of the drug and minimizes side effects.

One of the most important problems in oncology and cancer treatment is the development of drugs that cause the death of cancer cells, without damaging normal cells. Another problem to be solved is to suppress the drug resistance of cancer cells. The third important issue is to provide effective penetration of drug molecules to cancer cells. The recent advanced design of immunotoxins predicts the use of these immunotoxins as a potential therapeutic method to treat cancer patients. However, some limitations in the use of antibodies remain, such as tolerance, high molecular weight, and poor tissue penetration. Such limitations have led to the development of newer types of fusion proteins for therapeutic purposes.

Several investigations have shown that it is possible to produce immunospecific cytotoxins, including plant Ribosome-inactivating proteins (RIPs) and bacterial toxins, e.g. ricin, saporin, gelonin, and abrin, for the making the immunotoxin (IT) therapy.

RIPs are toxic N-glycosidases that depurate eukaryotic and prokaryotic rRNAs, thereby arresting protein synthesis during translation. Plant toxins are classified into 2 classes, Holotoxins, and Hemitoxins. Only the catalytic domains of both holotoxins and hemotoxins translocate to the cytosol, and the binding domains of holotoxins would be removed before translocation. These toxin-mediated processes stimulate the apoptotic pathway, leading to cell death. However, the translocation of plant toxins to the cytosol from the cell surface is still unknown.

In addition, RIPs are widely found in various plant species and within different tissues, but the mechanism of these effects is still not completely clear. Various RIPs have shown special properties including antibacterial, antifungal, antiviral, and insecticidal activity. Hence, in recent years, there has been considerable interest in RIPs due to their potential properties in the development of therapeutic agents such as toxin-antibody conjugates targeted against tumor cells.

As such, although existing techniques and agents are useful to some extent for some purposes, these prior efforts sometimes yield a poor therapeutic experienced by patients. Therefore, there is a need for effective therapeutic agents for targeting treatment. Also, there is a need for carrier vehicles to optimize the effect and reduce the toxic side effects of the drugs.

### SUMMARY OF THE INVENTION

The present invention discloses cancer treatment. The present invention uses targeted recombinant therapeutic protein as a chemotherapeutic agent to inhibit the growth of cancer cells.

According to the present invention, the targeted recombinant therapeutic protein comprises tumor-specific carrier vehicles delivering pharmacologic agents that are produced using recombinant DNA technology. The recombinant protein derived from Abrin-a A-chain able to inhibit the growth of cancer cells and conjugated to stxB to form Abrin-f-stxB.

In one embodiment, a conjugate for the delivery of a compound into cancer cells, comprising one or more modules including a first module, a second module and a third module, and a compound. The first module mediates cell targeting and facilitates cellular uptake. In one embodiment, the first module is a toxin protein or peptide having reduced or no toxicity and the module toxin protein is a Shiga toxin B-subunit peptide, an STx1a Shiga toxin B-subunit or an Abrin-a B-subunit. The second module facilitates transport to the endoplasmic reticulum (ER). In one embodiment, the second module is an oligopeptide with one or more of the amino acid sequence.

The third module mediates translocation from the ER to the cytosol. In one embodiment, the third module is a peptide, a protein, a C-terminal destabilizing oligopeptide or a C-terminal destabilizing oligopeptide that is an Stx1a Shiga toxin B-subunit, an Stx1b (VT1b) Shiga toxin B-subunit or a mutated Abrin-a A-subunit having reduced or no toxicity. In one embodiment, the compound is an antigen that is desired to be delivered to the cytosol.

The abrin toxin A chain and the Shiga toxin B chain are linked with the furin linker to form a recombinant therapeutic protein as a chemotherapeutic agent to inhibit the growth of cancer cells. In one embodiment, the abrin is a type II Ribosome-inactivating proteins (RIPs). The Shiga toxins (stxs) structural configuration comprises of an enzymatically active A-subunit (stxA) non-covalently linked with five identical B-subunits (stxB). In one embodiment, the stxB binds to the Gb3 and Gb4 receptors with comparable affinities. The furin is a type of protease and able to cut in R—X—[K/R]—R site in the endoplasmic network and Golgi apparatus.

In one embodiment, the peptide modules are chemically synthesized, by liquid phase or solid phase peptide synthesis, or the peptide is genetically engineered using recombinant DNA techniques and a cellular expression system, such as bacteria that includes Escherichia coli and yeast cells, insect cells, mammalian cells, etc., or an in vitro expression system.

In one embodiment, a method of constructing a recombinant protein, comprising the following steps. At one step, abrin-a A-chain is optimized based on the DNA sequence. At another step, non-toxic receptor-binding Shiga toxin subunit B stxB is inserted in the C-terminal side of the sequence and leads to functionalization of abrin-a A-chain as a catalytic chain of potent plant toxin. At another step, a furin linkage linker is placed between the Abrin-a A chain and stxB fragments for the separation of two sequences on both sides. At another step, Elastin-like peptide (ELP) with the length of 750 bp and Enterokinase cleavage site (En) with the length of 15 bp is used at the end of the gene construct in order to make it possible purification of the recombinant protein (Abrin-a A-chain:furin:stxB) from the rest of total proteins and subsequently release of ELP. At another step, internalization of Abrin-a A-chain into cells is mediated through a specific stxB-Gb3 interaction and Abrin-a A-chain is translocated to the cytosol. At another step, E. coli expression of optimized Abrin A-chain (Abrin) conjugated with stxB and ELP was carried out using gene cassette.

One aspect of the present disclosure is directed to a conjugate for the delivery of a compound into cancer cells, comprising: a first module that mediates cell targeting and facilitates cellular uptake; a second module that facilitates transport to the endoplasmic reticulum (ER); a third module that mediates translocation from the ER to the cytosol, and at least one compound, wherein the modules are covalently linked using a peptide linker module and is added with a furin cleavage site, wherein the furin will cleave the peptide linker at the furin cleavage site between two modules.

In one embodiment, the first module is a toxin protein or peptide having reduced or no toxicity and the module toxin protein is a Shiga toxin B-subunit peptide, an STx1a Shiga toxin B-subunit or an Abrin-a B-subunit. In another embodiment, the second module is an oligopeptide with one or more of the amino acid sequence. In one embodiment, the third module is a peptide, a protein, a C-terminal destabilizing oligopeptide or a C-terminal destabilizing oligopeptide that is an Stx1a Shiga toxin B-subunit, an Stx1b (VT1b) Shiga toxin B-subunit or a mutated Abrin-a A-subunit having reduced or no toxicity. In another embodiment, the compound is an antigen that is desired to be delivered to the cytosol.

In one embodiment, the abrin toxin A chain and the Shiga toxin B chain are linked with the furin linker to form a recombinant therapeutic protein as a chemotherapeutic agent to inhibit the growth of cancer cells. In another embodiment, abrin is a type II Ribosome-inactivating proteins (RIPs). In one embodiment, Shiga toxins (stxs) structural configuration comprises of an enzymatically active A-subunit (stxA) non-covalently linked with five identical B-subunits (stxB). In another embodiment, the stxB binds to the Gb3 and Gb4 receptors with comparable affinities. In one embodiment, the furin is a type of protease and able to cut in R—X—[K/R]—R site in the endoplasmic network and Golgi apparatus. In another embodiment, the peptide modules are chemically synthesized, by liquid phase or solid phase peptide synthesis, or the peptide is genetically engineered using recombinant DNA techniques and a cellular expression system, such as bacteria that includes Escherichia coli and yeast cells, insect cells, mammalian cells, etc., or an in vitro expression system.

Another aspect of the present disclosure is directed to a method of constructing a recombinant protein, comprising: optimizing abrin-a A-chain based on the DNA sequence; inserting non-toxic receptor-binding Shiga toxin subunit B stxB and enabling functionalization of abrin-a A-chain as a catalytic chain of potent plant toxin; placing furin linkage between the abrin-A chain and stxB fragments for the separation of tow sequences on both sides; purifying the recombinant protein from the rest of the total proteins and subsequently releasing ELP by using elastin-like peptide (ELP) and enterokinase cleavage site (En) at the end of the gene construct; mediating the internalization of abrin-a A-chain into cells via a specific stcB-Gb3 interaction and abrin-a A-chain is translocated to the cytosol; and conjugating E. coli expression of optimized abrin A-chain and stxB carried out using gene cassette.

In one embodiment, the furin cleavage allows the more facile release of the toxin from the complex once internalize by the endosomal compartment. In another embodiment, the method further utilizes one or more restriction sites including SalI, EcoRI, XhoI and BamHI for cloning purpose. In one embodiment, the high expression of the Gb3 at the surface of cancer cells is considered as the specific binding of Shiga toxin stxB to Gb3, in which stxB acts as a drug delivery system. In another embodiment, the gene cassette Abrin-f-stxB-En-ELP inserted to pET28a+ for the transformation of E. coli strain Bl21. One aspect of the present disclosure is directed to a method of treating cancer in a subject in need thereof, comprising administering to the subject a therapeutically effective amount of the antibody of claim 1. In one embodiment, the recombinant protein Abrin-f-stxB exhibits a high binding affinity of stxB as a drug delivery system to Gb3 receptor in targeted cancer diagnosis and treatment. In another embodiment, the cancer is colorectal and breast cancer.

Another aspect of the present disclosure is directed to a conjugate for the delivery of a compound into cancer cells, comprising: a first module that is a deadly toxin for the cell but does not have the ability to enter the cell; a second module that helps the first module connect to the surface of cancer cells and enter the cell; a third module, which allows easy and economical purification of recombinant chimeric protein, and at least one compound, wherein the modules are covalently linked using a peptide linker module and is added with a furin cleavage site, wherein the furin will cleave the peptide linker at the furin cleavage site between two modules.

In one embodiment, the first module (Abrin A Chain) is a toxin peptide having reduced or no toxicity and the second module (Shiga toxin B-subunit) is the delivery part of Shiga toxin that allows the toxin (Abrin A Chain) to penetrate into the cancer cell. In one embodiment, the second module is an oligopeptide with 69 amino acid sequences. In one embodiment, the third module is a peptide, a protein with 250 amino acid sequences, that is a tag for recombinant chimeric protein purification. In another embodiment, the compound is an antigen that is desired to be delivered to the cytosol of cancer cells.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF DRAWINGS

FIG. 1 exemplarily illustrates a schematic diagram of an abrin molecule, according to an embodiment of the present invention;

FIG. 2 exemplarily illustrates a schematic diagram of the abrin molecule leading to cell death, according to an embodiment of the present invention;

FIG. 3 exemplarily illustrates a schematic representation of a Shiga toxin (stxs), according to an embodiment of the present invention;

FIG. 4 exemplarily illustrates a schematic representation of gene construct used for bacterial transformation, according to an embodiment of the present invention;

FIG. 5 exemplarily illustrates an SDS-PAGE analysis of total bacterial and purified protein expressed in E. coli strain BL21 (DE3), according to an embodiment of the present invention;

FIG. 6 exemplarily illustrates various patterns of the recombinant bacterial protein, according to an embodiment of the present invention;

FIG. 7 exemplarily illustrates a process of separation and purification of Abrin-f-stxB from ELP after cleavage of the fusion protein, according to an embodiment of the present invention;

FIG. 8 exemplarily illustrates a graph of the anti-proliferative effects of Abrin-f-stxB on LS180 colorectal cancer cell line, according to an embodiment of the present invention;

FIG. 9 exemplarily illustrates a graph of the anti-proliferative effects of Abrin-f-stxB on MCF7 human breast cancer cell line, according to an embodiment of the present invention;

FIG. 10 exemplarily illustrates a graph of the anti-proliferative effect of Abrin-f-stxB on L929 normal cell line, according to an embodiment of the present invention;

FIG. 11 exemplarily illustrates a graph of the Apoptosis assay using flow cytometry on MCF7 human breast cell line, according to an embodiment of the present invention; and

FIG. 12 exemplarily illustrates a table having various drugs used to treat colon and breast cancer, according to an embodiment of the present invention.

### DETAILED DESCRIPTION

The present invention generally relates to the field of cancer treatment. More particularly, the present invention relates to targeted delivery of recombinant therapeutic protein as a chemotherapeutic agent to inhibit the growth of cancer cells.

A description of embodiments of the present invention will now be given with reference to the figures. It is expected that the present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

According to the present invention, the targeted recombinant therapeutic protein comprises tumor-specific carrier vehicles delivering pharmacologic agents that are produced using recombinant DNA technology. The recombinant protein derived from Abrin-a A-chain able to inhibit the growth of cancer cells and conjugated to stxB to form Abrin-f-stxB.

In one embodiment, a conjugate for the delivery of a compound into cancer cells, comprising one or more modules including a first module, a second module and a third module, and a compound. The first module mediates cell targeting and facilitates cellular uptake. In one embodiment, the first module is a toxin protein or peptide having reduced or no toxicity and the module toxin protein is a Shiga toxin B-subunit peptide, an STx1a Shiga toxin B-subunit or an Abrin-a B-subunit. The second module facilitates transport to the endoplasmic reticulum (ER). In one embodiment, the second module is an oligopeptide with one or more of the amino acid sequence. The third module mediates translocation from the ER to the cytosol. In one embodiment, the third module is a peptide, a protein, a C-terminal destabilizing oligopeptide or a C-terminal destabilizing oligopeptide that is an Stx1a Shiga toxin B-subunit, an Stx1b (VT1b) Shiga toxin B-subunit or a mutated Abrin-a A-subunit having reduced or no toxicity. In one embodiment, the compound is an antigen that is desired to be delivered to the cytosol.

The abrin toxin A chain and the Shiga toxin B chain are linked with the furin linker to form a recombinant therapeutic protein as a chemotherapeutic agent to inhibit the growth of cancer cells. In one embodiment, the abrin is a type II Ribosome-inactivating proteins (RIPs). The Shiga toxins (stxs) structural configuration comprises of an enzymatically active A-subunit (stxA) non-covalently linked with five identical B-subunits (stxB). In one embodiment, the stxB binds to the Gb3 and Gb4 receptors with comparable affinities. The furin is a type of protease and able to cut in R—X—[K/R]—R site in the endoplasmic network and Golgi apparatus.

In one embodiment, the peptide modules are chemically synthesized, by liquid phase or solid phase peptide synthesis, or the peptide is genetically engineered using recombinant DNA techniques and a cellular expression system, such as bacteria that includes Escherichia coli and yeast cells, insect cells, mammalian cells, etc., or an in vitro expression system.

In one embodiment, a method of constructing a recombinant protein, comprising the following steps. At one step, abrin-a A-chain is optimized based on the DNA sequence. At another step, non-toxic receptor-binding Shiga toxin subunit B stxB is inserted in the C-terminal side of the sequence and leads to functionalization of abrin-a A-chain as a catalytic chain of potent plant toxin. At another step, a furin linkage linker is placed between the Abrin-a A chain and stxB fragments for the separation of two sequences on both sides. The furin cleavage allows the more facile release of the toxin from the complex once internalizes by the endosomal compartment.

At another step, Elastin-like peptide (ELP) with the length of 750 bp and Enterokinase cleavage site (En) with the length of 15 bp is used at the end of the gene construct in order to make it possible purification of the recombinant protein (Abrin-a A-chain:furin:stxB) from the rest of total proteins and subsequently release of ELP. At another step, the internalization of Abrin-a A-chain into cells is mediated through a specific stxB-Gb3 interaction and Abrin-a A-chain is translocated to the cytosol. The high expression of the Gb3 at the surface of cancer cells are considered as the specific binding of Shiga toxin stxB to Gb3, in which stxB acts as a drug delivery system. At another step, E. coli expression of optimized Abrin A-chain (Abrin) conjugated with ELP and stxB was carried out using gene cassette. In one embodiment, the method further utilizes one or more restriction sites including SalI, EcoRI, XhoI, and BamHI for cloning purposes.

In one embodiment, a method of treating cancer in a subject in need thereof comprising administering to the subject a therapeutically effective amount of the antibody. The recombinant protein Abrin-f-stxB exhibits a high binding affinity of stxB as a drug delivery system to Gb3 receptor in targeted cancer diagnosis and treatment. In one embodiment, the cancer is colorectal and breast cancer.

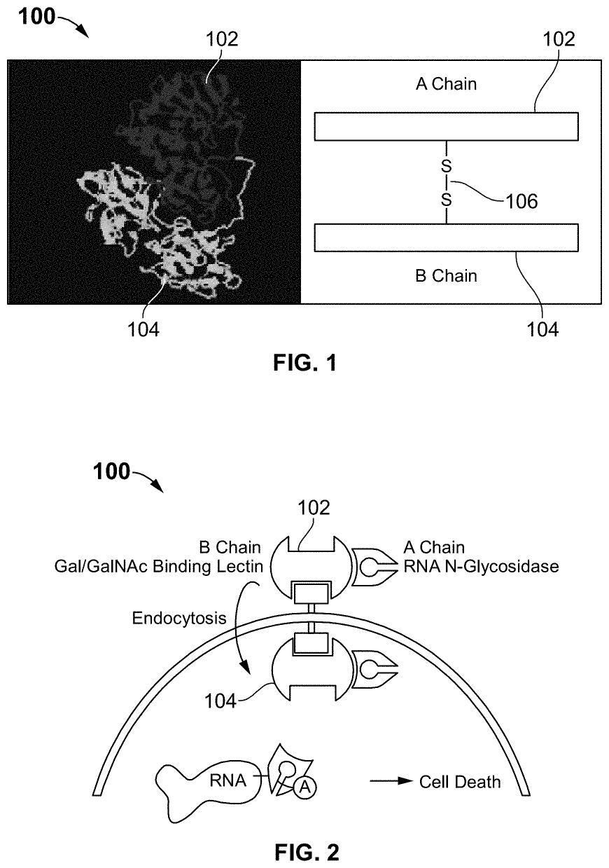

Referring to FIG. 1, a schematic diagram of an abrin molecule 100, according to one embodiment of the present invention. In one embodiment, abrin is a potent plant-derived toxin. Abrin is isolated from the seed of the tropical plant Abrus precatorius L. Abrin is a type of heterodimeric cytotoxin and a member of the Ribosome-inactivating proteins (RIPs)-type II family with different two domains, including a catalytically toxic A-chain 102, (˜30 kDa), and a lectin-like B-chain (˜32 kDa) 104. The catalytically toxic A-chain 102 and the lectin-like B-chain 104 are joined together by a single disulfide bond 106.

Referring to FIG. 2, a schematic diagram of the abrin molecule 100 leading to cell death, according to one embodiment of the present invention. The abrin molecule 100 has the toxic A-chain 102 and the lactin-like B-chain 104. The A-chain 102 has N-glycosidase activity in eukaryotic 28S rRNA. The N-glycosidase activity may catalyze depuration, which is a single adenine residue (A4324 site) located near the 30 terminal ends of the 28S rRNA within the a-sarcin/ricin loop, a highly conserved sequence found of the 60S large subunit of eukaryotic ribosomes. This activity prevents the formation of a critical stem-loop configuration which results in the inability of the ribosome to bind elongation factor-2, thereby causing the translational arrest of eukaryotic protein synthesis at the translocation step. The activity subsequently leads to cell death.

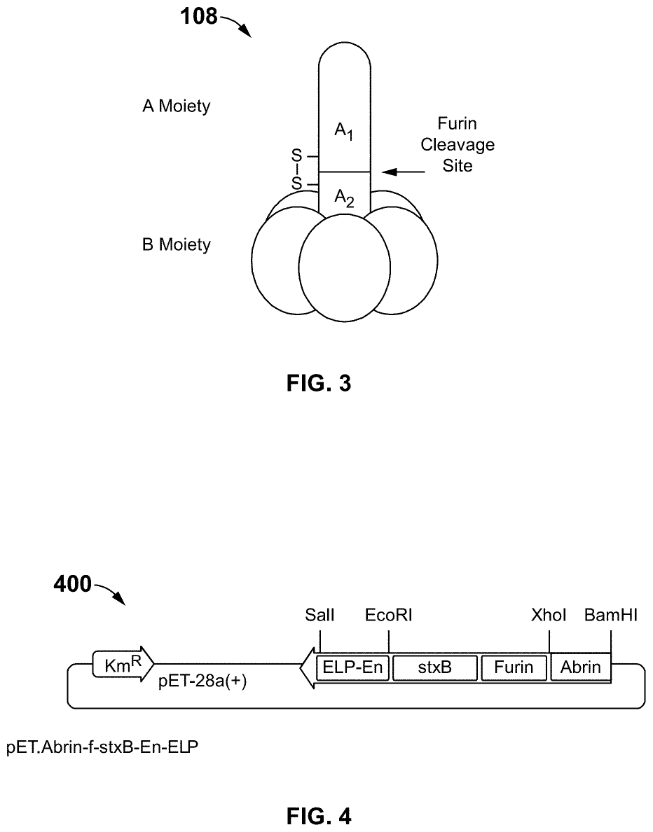

Referring to FIG. 3, a schematic representation of a Shiga toxin (stxs) 108, according to one embodiment of the present invention. Abrin is one of the most lethal types of the toxin, which is known to be 75 times more potent than ricin with an LD50 (50% lethal dose) values of only 2.8 μg/kg body weight of mice. There are four isoforms of abrin that have been isolated, including the abrin-a, abrin-b, abrin-c and abrin-d. The abrin-a isomer is the most toxic and abrin-b has been known to have the least cytotoxic potency. Previous investigations showed that Abrin-a A-chain has anticancer activity against some kinds of cancers. Therefore, RIPs are potent weapon candidates for use in immunotherapy of various diseases, including cancer. In one embodiment, targeting abrin-a A-chain could be a valuable anticancer drug.

In one embodiment, the Shiga toxin (stxs) 108 has one or more target moieties. The target moiety facilitates the transfer and internalization of RIPs into cancer cells. For this purpose, various bacterial toxins have been used in Recombinant immunotoxins (RITs) for targeting cancer cells. All members of the Shiga toxins (stxs) family share the same structural configuration comprised of an enzymatically-active A-subunit (stxA) non-covalently linked with five identical B-subunits (stxB). Thus Stxs 108 are part of a larger class of structurally-conserved bacterial protein toxins, termed AB₅ toxins.

### Example

There are numerous studies have demonstrated that in addition to being potent protein synthesis inhibitors, stxs are also multifunctional proteins capable of activating multiple cell stress signaling pathways, which may result in apoptosis, autophagy or activation of the innate immune response. According to several researches on Abrin-a A-chain (abrin) and stxB, it is desired to examine chimeric protein Abrin-f-stxB (in which f means furin linker) as a targeted recombinant therapeutic protein. Furin is a type of protease and able to cut in R—X—[K/R]—R site in the endoplasmic network and Golgi apparatus. Furin linkage linker (RRKR) was placed between Abrin and stxB in order to make it possible separation of tow sequences on both sides.

In order to express protein and analyzing its features, bacterial protein expression s programmed in Escherichia coli (DE3) as a suitable host for expressing in a rapid growth rate, high levels of protein expression and cost-effectiveness in comparison with other expression systems. In addition, to significantly improve protein expression in E. coli, the codon-optimization of interested DNA was done based on the codon preferences in the host. Abrin-a A-chain was optimized based on the corresponding DNA sequence (Genbank Accession No. X76720). Recombinant protein purification is an important downstream process, so a thermally responsive elastin-like polypeptides (ELPs) as a highly efficient protein Non-chromatographic purification method, termed Inverse Transition Cycling (ITC), was used to purify proteins from Escherichia coli. This strategy is not restricted to the equipment; hence they can be carried out cheap and simple, rapid and also efficient.

Further, the ELP tag is applied for the purification of the recombinant proteins for the successful separation of the target protein from the solution. The ELP tag is composed of about 10-250 repeated motifs (VPGXG). The X could be any amino acid except proline. The length of ELP and the composition of each motif affect its ability for protein purification. The ELP tags have been successfully used for purification of several target protein-ELP fusions without column chromatography from E. coli, sortase A (SrtA) transpeptidase from Staphylococcus aureus, thioredoxin (TRX), soluble murine tumor necrosis factor α (TNFα), chloramphenicol acetyltransferase, calmodulin, blue fluorescent protein, β-galactosidas], and α-amylase from Bacillus licheniformis.

For purification purpose, an optimized abrin-a A-chain-StxB is tagged with ELP containing about 50 repeats of the pentapeptide sequence Val-Pro-Gly-Val-Gly according to E. coli expression system codon preferences. This chimeric protein successfully expressed in the host and after ELP-based purification, the ELP tag was enzymatically separated from the rest of the chimeric protein. Afterward, the purified (Abrin-f-stxB-En-ELP) fusion was cleaved with enterokinase protease and examined to test if it is effective on suppressing cancerous cells. The anti-proliferative effects of Abrin-f-stxB were carried out on cells including, but not limited to, LS180 colorectal line, MCF7 human breast cancer cell line, and L929 normal cell line. The cell viability was measured by the MTT assay. Cell lines were exposed to different concentrations of Abrin-f-stxB. Doxorubicin (Doxo) as a common drug is used in chemotherapy and the percent of viable cells was calculated after 72 hours post-treatment.

According to the MTT assay results, the value of IC50 for Abrin-f-stxB on the LS180 colorectal cancer cell line is estimated less than about 0.001 and the IC50 of Abrin-f-stxB on the MCF7 human breast cancer cell line is estimated less than about 0.009 μg/ml (IC50 of Doxo estimated 0.03 μg/ml). The results indicate no significant activity in L929 normal cells (IC50 more than 50 μg/mL).

The results also indicate that the interested protein successfully suppressed the proliferation of cancer cells with no impact on normal cells. In addition, apoptosis assay was conducted using flow cytometry on MCF7 human breast cells (2*10⁶) treated with 25 ug/100 ul of recombinant protein Abrin-f-stxB followed by 24 h incubation. These results indicated that protein abrin-f-stxB induced apoptosis in MCF7 human breast cells.

Referring to FIG. 4, a schematic representation of gene construct 400 used for bacterial transformation, according to one embodiment of the present invention. The gene construct 400 comprises abrin, stxB, furin, and En-ELP. The Abrin (753 bp) is the multi-optimized sequence of Abrin-a A-chain. The stxB (207 bp) is Shiga toxin subunit B that is considered to be inserted in the C-terminal side of the sequence to increase the immunogenicity efficacy of Abrin-a A-chain. The Furin (12 bp) is a furin linkage linker, which is placed between Abrin and stxB to make it possible separation of tow sequences on both sides. The En-ELP (765 bp) is Elastin-liked peptide (ELP) with the length of about 750 bp plus Enterokinase cleavage site (En) with the length of about 15 bp has been used at the end of the gene construct to make it possible purification of the recombinant protein (Abrin-a A-chain:furin:stxB) from the rest of total proteins and subsequently release of ELP. The construct has been named pET. Abrin-f-stxB-En-ELP. Further, one or more restriction sites such as SalI, EcoRI, XhoI and BamHI are used for cloning purposes.

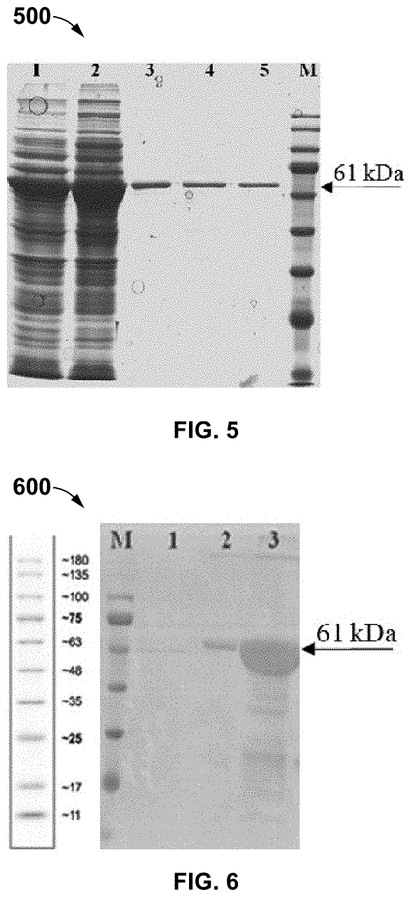

Referring to FIG. 5, a SDS-PAGE analyses 500 of total bacterial and purified protein expressed in E. coli strain BL21 (DE3), according to one embodiment of the present invention. The SDS-PAGE analysis 500 comprises one or more lanes including Lane-1, Lane-2, Lane-3, Lane-4, and Lane-5. Lanes 1 and 2 indicate total protein extracted from recombinant BL21 (DE3) carrying pET. Abrin-f-stxB-En-ELP plasmid. Lanes 3, 4, and 5 show ELP-based purification of protein Abrin-f-stxB-En-ELP. The SDS-PAGE analysis 500 further comprises Lane-M, which is a molecular weight protein size marker.

FIG. 6 exemplarily illustrates various patterns 600 of the recombinant bacterial protein, according to one embodiment of the present invention. The patterns 600 comprises one or more lanes including Lane-1, Lane-2, Lane-3, and Lane-M. Lane-1 has the total protein extracted from non-recombinant E. coli Bl21 (DE3) as control. Lane-2 has purified protein Abrin-f-stxB-En-ELP that shows interaction with anti-Abrin polyclonal antibody. Lane-3 shows the western blot patterns of the recombinant bacterial protein Abrin-f-stxB-En-ELP probed with polyclonal antibody against Abrin. Lane-1 shows the western blot patterns of the recombinant bacterial protein Abrin-f-stxB-En-ELP probed with polyclonal antibody against Abrin. Lane-2 has the total protein extracted from non-recombinant E. coli Bl21 (DE3) as control. Lane-3 has purified protein Abrin-f-stxB-En-ELP that shows interaction with anti-Abrin polyclonal antibody. Lane-M has the total protein extracted from BL21 (DE3) carrying pET. Abrin-f-stxB-En-ELP plasmid, which is the molecular weight protein size marker.

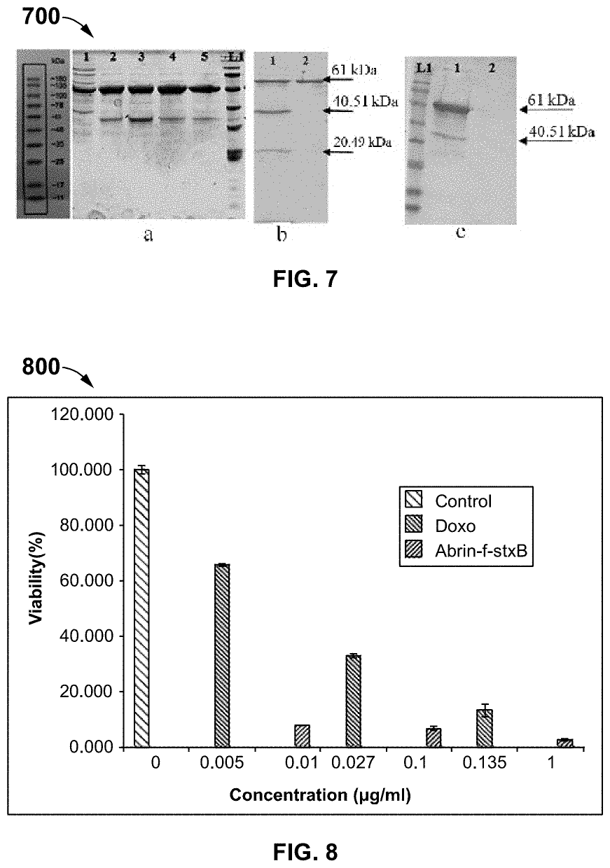

Referring to FIG. 7, a process 700 of separation and purification of Abrin-f-stxB from ELP after cleavage of the fusion protein, according to one embodiment of the present invention. The process 700 comprises the following steps. At step (a), Abrin-f-stxB is separated from ELP after cleavage of the fusion protein by enterokinase through electrophoresis on 10% SDS-PAGE analysis. It has four lanes including Lane-1, Lane-2, Lane-3, Lane-4, and Lane-5. The Lanes 1, 2, 3, 4 and 5 indicate the purified Abrin-f-stxB-En-ELP (61 kDa) fusion is cleaved with enterokinase protease and produces the purified protein Abrin-f-stxB (40.51 kDa). At step (b), SDS-PAGE analysis performs blue silver staining. It has two lanes including Lane-1 and Lane-2.

Lane-1 indicates purified protein Abrin-f-stxB-En-ELP and the cleavage products of digestion with enterokinase including the purified protein Abrin-f-stxB and ELP tag (20.49 kDa). Lane-2 shows the purified Abrin-f-stxB-En-ELP. At step (c), creates western blot patterns of the purified protein Abrin-f-stxB from ELP after cleavage of protein Abrin-f-stxB-En-ELP by enterokinase. It has three lanes including Lane-1, Lane-2, and Lane-L1. Lane-1 indicates interaction with anti-abrin polyclonal antibody in purified proteins Abrin-f-stxB-En-ELP and Abrin-f-stxB. Lane-2 shows total protein extracted from non-recombinant E. coli Bl21 (DE3) as control. Lane-L1 shows protein ladder (Sinaclon Iran). Arrowheads indicate the Abrin-f-stxB-En-ELP and the cleavage products of digestion with enterokinase.

Results:

Referring to FIG. 8, a graph 800 of the anti-proliferative effects of Abrin-f-stxB on LS180 colorectal cancer cell line, according to one embodiment of the present invention. The anti-proliferative effects of Abrin-f-stxB on LS180 colorectal cancer cell line. The cell viability was measured by the MTT assay. LS180 cells were exposed to different concentrations of Abrin-f-stxB. Also, LS180 cells were exposed to different drug concentrations of Doxorubicin (Doxo) and percent of viable cells was calculated after 72 hours post-treatment. Doxo is considered as positive control. Doxo is a common drug sing in chemotherapy. The value of IC50 for Doxo and Abrin-f-stxB was measured 0.03 and 0.001 ug/ml, respectively. The results are means±standard deviation (SD).

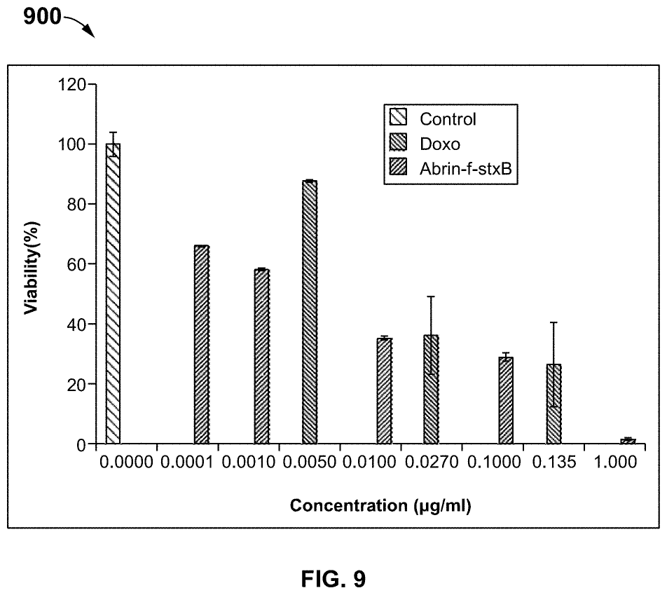

Referring to FIG. 9, a graph 900 of the anti-proliferative effects of Abrin-f-stxB on MCF7 human breast cancer cell line, according to one embodiment of the present invention. The cell viability was measured by the MIT assay. LS180 cells were exposed to different concentration of Abrin-f-stxB. Also, MCF7 cells were exposed to different drug concentration of Doxo and the percent of viable cells was calculated after 72 hours post treatment. Doxo is considered as positive control. Doxo is a common drug using in chemotherapy. The value of IC50 for Doxo and Abrin-f-stxB is measured 0.03 and 0.009 μg/ml, respectively. The results are means±standard deviation (SD).

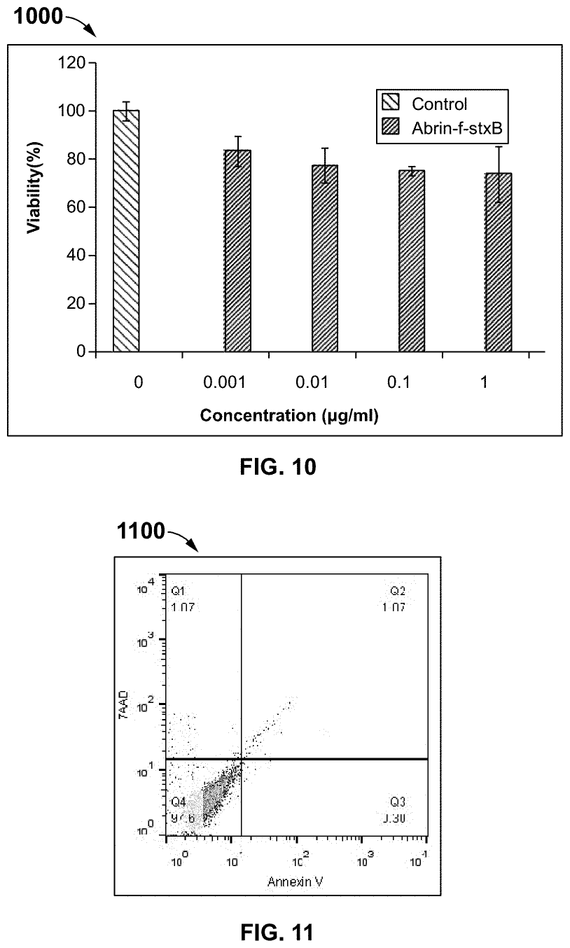

Referring to FIG. 10, a graph 1000 of the anti-proliferative effect of Abrin-f-stxB on L929 normal cell line, according to one embodiment of the present invention. Dose-response results of Abrin-f-stxB recombinant protein on L929 normal cells. Cells were cultured in 96-well plates and treated with indicated concentrations of Abrin-f-stxB. Cell viability was measured by MIT assay. The value of IC50 for L929 normal cells was measured more than 50 μg/mL. Data represent the mean±SD of three independent experiments.

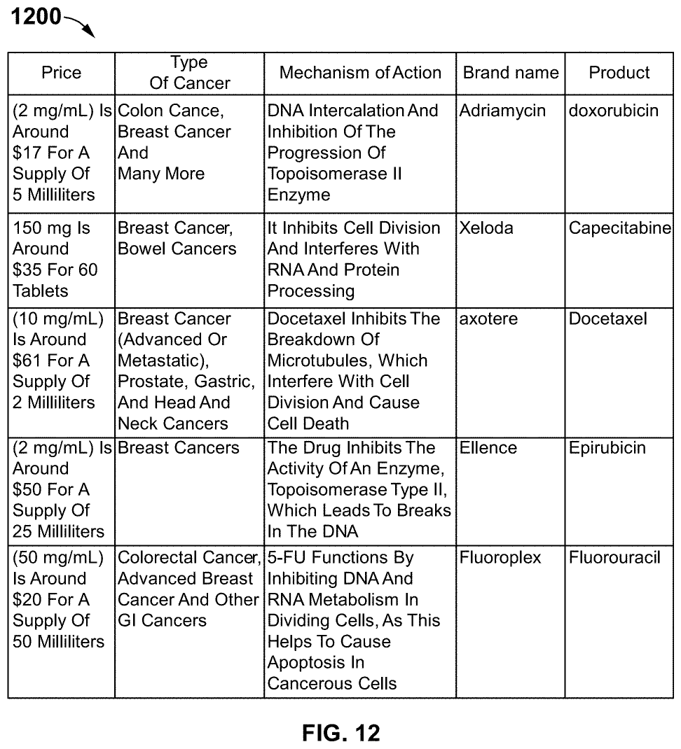

Referring to FIG. 11, a graph 1100 of the Apoptosis assay using flow cytometry on MCF7 human breast cell line, according to one embodiment of the present invention. The evaluation is performed using representative flow cytometry scatter plots of 7AAD (Y axis) against Annexin-V-PE (X axis). Evaluation by Annexin V-PE versus 7AAD staining of MCF7 cells (2*106) treated with 25 ug/100 ul of recombinant protein Abrin-f-stxB was done after 24 h incubation. Apoptotic cell death of treated cells was detected by dual staining with Annexin V-PE and 7AAD followed by flow cytometric analysis. The bottom left (Q4) indicates living cells, the bottom right indicates (Q3) shows early apoptotic, the top right (Q2) is late apoptotic and the top left (Q1) is necrotic cells. The numbers indicate the respective percentages of total cell populations. Further, FIG. 12 shows a table 1200 having various drugs used to treat colon and breast cancer.

According to the present invention, the recombinant protein derived from Abrin-a A-chain can inhibit the growth of cancer cell lines in a very low dose without side effects. Using the non-toxic receptor-binding stxB leads to functionalization of Abrin-a A-chain as a catalytic chain of potent plant toxin. The internalization of Abrin-a. A-chain into cells is mediated through a specific stxB-Gb3 interaction and Abrin-a A-chain is translocated to the cytosol. In addition, furin cleavage linker is used between the Abrin-a A chain and stxB fragments. The furin cleavage would allow the more facile release of the toxin from the complex once internalized by the endosomal compartment. According to the MTT assay results, the IC50 of Abrin-f-stxB on the LS180 colorectal cancer cell line estimated less than 0.001, and the IC50 of Abrin-f-stxB on the MCF7 human breast cancer cell line estimated less than 0.009 μg/ml (IC50 of doxorubicin estimated 0.03 μg/ml). Results also indicated no significant activity in L929 normal cells (IC50 more than 50 μg/mL).

Advantageously, the recombinant protein Abrin-f-stxB exhibited a stronger anti-cancer effect than doxorubicin (Doxorubicin is a cytotoxic chemotherapy drug). The treatment of LS180 and MCF7 cancer cells with Abrin-f-stxB significantly, suppressed cell proliferation and induced cell apoptosis. The recombinant protein Abrin-f-stxB as an agent that can selectively induce cell death without affecting the normal cells will be an ideal chemotherapeutic agent against cancer cells.

Abrin-f-stxB could have potential for the treatment of colorectal and breast cancer cells. The protein Abrin-f-stxB exhibits a high binding affinity of stxb as a drug delivery system to Gb3 receptor in targeted cancer diagnosis and treatment. The results strongly demonstrate that Abrin-a A-chain is internalized into cancer cells in a receptor-dependent manner. The recombinant protein Abrin-f-stxB is a powerful drug for killing cancer cells.

The foregoing description comprise illustrative embodiments of the present invention. Having thus described exemplary embodiments of the present invention, it should be noted by those skilled in the art that the within disclosures are exemplary only, and that various other alternatives, adaptations, and modifications may be made within the scope of the present invention. Merely listing or numbering the steps of a method in a certain order does not constitute any limitation on the order of the steps of that method.

Many modifications and other embodiments of the invention will come to mind to one skilled in the art to which this invention pertains having the benefit of the teachings presented in the foregoing descriptions. Although specific terms may be employed herein, they are used only in generic and descriptive sense and not for purposes of limitation. Accordingly, the present invention is not limited to the specific embodiments illustrated herein. While the above is a complete description of the preferred embodiments of the invention, various alternatives, modifications, and equivalents may be used. Therefore, the above description and the examples should not be taken as limiting the scope of the invention, which is defined by the appended claims.

1. A conjugate for the delivery of a compound into cancer cells, comprising:
a first module that is a deadly toxin for the cell but does not have the ability to enter the cell;
a second module that helps the first module to connect to the surface of cancer cells and enter the cell;
a third module, which allows easy and economical purification of recombinant chimeric protein, and at least one compound,
wherein the modules are covalently linked using a peptide linker module and is added with a furin cleavage site,
wherein the furin will cleave the peptide linker at the furin cleavage site between two modules.

2. The conjugate of claim 1, wherein the first module (Abrin A Chain) is a toxin peptide having reduced or no toxicity and the second module (Shiga toxin B-subunit) is the delivery part of Shiga toxin that allows the toxin (Abrin A Chain) to penetrate into the cancer cell.

3. The conjugate of claim 1, wherein the second module is an oligopeptide with 69 amino acid sequences.

4. The conjugate of claim 1, wherein the third module is a peptide, a protein with 250 amino acid sequences, that is a tag for recombinant chimeric protein purification.

5. The conjugate of claim 1, wherein the compound is an antigen that is desired to be delivered to the cytosol of cancer cells.

6. The conjugate of claim 1, wherein the abrin toxin A chain and the Shiga toxin B chain are linked with the furin linker to form a recombinant therapeutic protein as a chemotherapeutic agent to inhibit the growth of cancer cells.

7. The conjugate of claim 1, wherein abrin is a type II Ribosome-inactivating proteins (RIPs).

8. The conjugate of claim 1, wherein Shiga toxins (stxs) structural configuration comprises of an enzymatically active A-subunit (stxA) non-covalently linked with five identical B-subunits (stxB).

9. The conjugate of claim 1, wherein the stxB binds to the Gb3 and Gb4 receptors with comparable affinities.

10. The conjugate of claim 1, wherein the furin is a type of protease and able to cut in R—X—[K/R]—R site in the endoplasmic network and Golgi apparatus.

11. The conjugate of claim 1, wherein the peptide modules are chemically synthesized, by liquid phase or solid phase peptide synthesis, or the peptide is genetically engineered using recombinant DNA techniques and a cellular expression system, such as bacteria that includes Escherichia coli and yeast cells, insect cells, mammalian cells, etc., or an in vitro expression system.

12. A method of constructing a recombinant protein, comprising:
optimizing abrin-a A-chain based on the DNA sequence;
inserting non-toxic receptor-binding Shiga toxin subunit B stxB and enabling functionalization of abrin-a A-chain as a catalytic chain of potent plant toxin;
placing furin linkage between abrin-A chain and stxB fragments for the separation of tow sequences on both sides;
purifying the recombinant protein from the rest of the total proteins and subsequently releasing ELP by using elastin-like peptide (ELP) and enterokinase cleavage site (En) at the end of the gene construct;
mediating the internalization of abrin-a A-chain into cells via a specific stcB-Gb3 interaction and abrin-a A-chain is translocated to the cytosol, and
conjugating E. coli expression of optimized abrin A-chain and stxB carried out using gene cassette.

13. The method of claim 12, wherein the furin cleavage allows more facile release of the toxin from the complex once internalize by the endosomal compartment.

14. The method of claim 12, further utilizes one or more restriction sites including SalI, EcoRI, XhoI and BamHI for cloning purpose.

15. The method of claim 12, wherein the high expression of the Gb3 at the surface of cancer cells is considered as the specific binding of Shiga toxin stxB to Gb3, in which stxB acts as a drug delivery system.

16. The method of claim 12, wherein the gene cassette Abrin-f-stxB-En-ELP inserted to pET28a+ for the transformation of E. coli strain Bl21.

17. A method of treating cancer in a subject in need thereof comprising administering to the subject a therapeutically effective amount of the antibody of claim 1.

18. The method of claim 17, wherein the recombinant protein Abrin-f-stxB exhibits a high binding affinity of stxB as a drug delivery system to Gb3 receptor in targeted cancer diagnosis and treatment.

19. The method of claim 17, wherein the cancer is colorectal and breast cancer.
